(19) United States
(12) Patent Application Publication
(10) Pub. No.: US 2014/0257155 A1
(43) Pub. Date: Sep. 11, 2014

(54) Kinesiologic Tape

(71) Applicants: Gazi Altinok, Remscheid (DE); Stefanos Vafiadis, Hagen (DE); Tesfaldet Zemichael, Dortmund (DE); Abidin Celik, Remscheid (DE)

(72) Inventors: Gazi Altinok, Remscheid (DE); Stefanos Vafiadis, Hagen (DE); Tesfaldet Zemichael, Dortmund (DE); Abidin Celik, Remscheid (DE)

(21) Appl. No.: 14/117,694

(22) PCT Filed: May 14, 2012
(86) PCT No.: PCT/EP2012/058846

§ 371 Date: May 24, 2014

(30) Foreign Application Priority Data
May 17, 2011 (DE) 202011100806.5
Jul. 19, 2011 (DE) 102011079388.7
Sep. 29, 2011 (DE) 102011083681.0
Dec. 28, 2011 (EP) 11195854.2

(51) Int. Cl. A61F 5/40

(52) U.S. Cl. CPC A61F 5/40
USPC 602/1

(57) ABSTRACT

The present invention relates to a kinesiologic tape which is provided with at least one molded body, said molded body comprising metal, healing stones, wood, ceramics and/or combinations thereof.

The present invention relates to a kinesiologic tape which is provided with at least one molded body, said molded body comprising metal, healing stones, wood, ceramics and/or combinations thereof.

Kinesiologic tapes are especially known in sports medicine and orthopedics. They are flexible plasters with elastic properties, which are adjusted to those of the human muscle. This results in no restrictions of movement of the patient. Kinesiologic tapes exhibit a targeted impact on the nervous and metabolic system of the patient. By applying different adhesive techniques, the therapist achieves, for example, for bruises, overused joints or muscle problems a clearly noticeable and, above all, fast reduction of pain. Furthermore, it also leads to a sustained improvement in muscle function and support of joints. By the tape a specific lifting of certain parts of the skin occurs, which in addition stimulates the lymphatic system. Even positive effects on internal organs have been described. Then, particularly different application techniques are used in combination with the pre-stretching of the tape.

In the current adhesive techniques, mainly a stimulus transmission occurs via the glued tissue. Due to the nature of the tapes, these stimuli are mostly pronounced flat and therefore not specifically limited to a single muscle portion.

The classical acupuncture uses needles in specific skin sections, which are associated with a particular organ or nerve, and which are inserted into the subcutaneous tissue, achieving a calming, strengthening and restoring of the function of the organ or the affected nerve. It is however proven that even the acupressure can be specifically used in the treatment of diseases or pain. Therein, an external—usually targeted punctual—pressure is directed on the body, which results in a healing influence on certain areas. If there is a specific punctual pressure, then the pressure-loaded area is designated as a reflex point. However, acupressure requires a constant external pressure, which is directed to the body. Therefore, prolonged manual acupressure treatments are usually very tiring for the therapist.

DE 42 40 952 A1 describes a device for the specific application of acupressure, which allows application of a punctual pressure-load over a prolonged period. Therefore, a dressing is used, which is formed so that it comprises elevations or knobs or nipples at predetermined locations. These consist of plastic or a high-density textile fabric. Similarly DE 7934840 U1 describes a device for zone treatment, wherein a wound plaster-like carrier with a non-stretchable area, onto which a hard elevation is applied, is used. Also EP 0779068 A1 describes a health-promoting device with acupressure effect, which consists of a carrier with elevations. Therein, the elevations are formed of silicone with appropriate elasticity.

In contrast EP 0081109 A2 describes a medicinal plaster for the treatment of larger areas, wherein permanent magnets are applied to this plaster. They are embedded in the plaster in a way, that a pressureless application to the skin is possible.

Thus, on the one hand a kinesiologic tape is known from the prior art, which has the characteristics for stimulating and stimulus application of parts of the body-surface, exercised by its nature. On the other hand, the application of elevations below usually non-stretchable carriers for exerting pressure impact on specific reflex zones is known.

A disadvantage of the previously known systems is that kinesiologic tapes can only be used as an accompanying treatment to relieve pain. With such tapes no reflex points can be stimulated.

The known acupressure devices, however, have the disadvantage that only selective stimuli can be set. A stimulation of the whole metabolism by stimulating the subcutaneous tissue, muscle fascia and the muscle tissue is not possible with the mentioned acupressure devices. Furthermore, these devices cannot be adjusted individually to the affected body region, and especially not to the individual patient.

In addition, both therapy methods have the disadvantage that a supportive heat or cryotherapy of the concerned body parts is limited.

The heat-insulating effect of kinesiologic tapes on the one hand but also of the elevations of the acupressure devices on the other hand prevent heat or cold transfer to the glued/pressure-loaded and therefore to the regions to be treated.

Starting from this prior art, the object of the present invention is to provide a kinesiologic tape, which is at the same time suitable for applying pressure with acupressure effect on the glued part of the body.

Here, the present invention shall preferably exhibit an increased analgesic effect and the enhanced stimulation of blood circulation, the tone regulation of muscle and of the venous/lymphatic reflux.

Moreover, the present invention shall provide a kinesiologic tape, which preferably allows a concomitant punctual pressure application with acupressure effect on the glued part of the body.

Furthermore, the invention shall preferably allow an adjustment of the optionally punctual pressure on the needs of the individual patient. This shall particularly allow an individualization to each disease, the resulting body part to be treated and the particular body shape of the individual patient.

In a further aspect of the present invention, the kinesiologic tape shall preferably also be suitable in supporting heat therapy.

In a further aspect of the present invention the kinesiologic tape shall preferably also be suitable in supporting cryotherapy.

In a further aspect of the present invention, the kinesiologic tape shall preferably also be suitable in supporting vibration massage.

These objects are solved by providing a kinesiologic tape with at least one molded body.

In a first aspect of the present invention, this kinesiologic tape with at least a molded body is characterized in that the at least one molded body comprises a material which is selected from the group consisting of metals, healing stones, wood, ceramics and/or combinations thereof. It has been found that the use of a kinesiologic tape in combination with at least one molded body exhibits particularly good analgesic properties and beneficial effects can be observed with respect to the healing of the patient.

Surprisingly, it was found that an analgesic effect of the tape on one hand and a healing acupressure effect on the other hand can be exceeded by the inventive kinesiologic tape in a supra-additive manner.

In a second aspect of the present invention, the at least one molded body consists of metal, healing stones, wood, ceramics and/or combinations thereof.

In a further aspect of the present invention, the at least one molded body is provided on the side of the kinesiologic tape which is to be glued to the body site.

In another aspect, the at least one molded body is such that it can exert pressure on at least one reflex point. In the context of the present invention a reflex point is understood to be the area, which is covered by a molded body on a body portion. This term is used interchangeably with the well-known terms reflex zone, trigger point and connective tissue zone used in the literature. Preferably, pressure is exerted onto this reflex point by the at least a molded body. In an embodiment of the present invention by the application of pressure via the at least one molded body a corresponding internal organ can be stimulated.

In a further embodiment the at least one molded body is formed such that a planar pressure is applied to a reflex point. For this purpose, the at least one molded body exhibits a flat shape. This flat shape can preferably also exhibit a curvature. In another embodiment, said at least one molded body is formed such that a punctual pressure is applied to a reflex point. In this case, a pointed form of the at least one molded body is preferred.

The term “at least one molded body” is understood within the present invention that one or more uniform molded bodies, one or more non-uniform molded bodies, one or molded bodies of identical materials, one or more molded bodies of different materials and/or combinations thereof can be used.

In a further embodiment the kinesiologic tape exhibits two or more molded bodies, which are such that at the same time a punctual and planar pressure can be applied on two or more reflex points.

In a further aspect of the present invention a kinesiologic tape is provided wherein at least one molded body for pressure application on at least one reflex point is present on the side facing the body site to be glued, wherein the at least one molded body comprises a material which is selected from the group consisting of metals, healing stones, wood, ceramics and/or combinations thereof. Preferably, metals are used as the material for the at least one molded body. In a preferred embodiment these may also have magnetic properties. Especially preferred is the use of copper as the material for the at least one molded body. Copper has a high thermal conductivity. This makes it particularly suitable for a specific heat and/or cryotherapy. Furthermore, this material is said to have properties which support the healing of a patient.

In a preferred embodiment the material of the at least one molded body has a high thermal conductivity of 10-430 W/(m·K), preferably 100 to 410 W/(m·K), more preferably 200 to 400 W/(m·K), even more preferred 240-380 W/(m·K).

In this case it is preferred to provide a kinesiologic tape with at least one molded body having a shape selected from the group consisting of sphere, sphere chain, ring, hemisphere, ellipsoid, rotational ellipsoid, paraboloid, rotational paraboloid, hyperboloid, oloid, polyhedron, tetrahedron, cube, cuboid, octahedron, icosahedron, dodecahedron, cuboctahedron, rhombic dodecahedron, icosidodecahedron, rhombic triaktontahedron, deltahedron, pyramid, prism, antiprism, truncated pyramid, torus, cylinder, cone, truncated cone, geodesic dome and/or combinations thereof. The at least one molded body can be formed or applied to the tape so that optionally at least one surface of the molded body bears on at least one reflex point, wherein the surface is selected from the group consisting of rectangle, square, triangle, rhombus, circle, ellipse, oval and/or combinations thereof. In a preferred embodiment a circular molded body is used, which has a slight curvature. The curvature is preferably facing the glued reflex point. Preferably, by the so-shaped embodiment a particularly good friction massage of the reflex point can be realized.

In a preferred embodiment the kinesiologic tape comprises two or more molded bodies of the same and/or different size. These two or more molded bodies are arranged on the tape so that a triangle, optionally with further molded bodies of the same or different size in the area surrounded by the triangle, a circle, optionally with further molded bodies of the same or different size in the area surrounded by the circle, a spiral, a star, a line, a rectangle, optionally with further molded bodies of the same or different size in the area surrounded by the rectangle, a cross, an X-shape, a C-shape, a T-shape, an L-shape, an Y-shape, optionally with further lines and/or an S-shape, optionally also mirror-inverted, is formed and these shapes are optionally formed by a plurality of parallel arranged molded bodies and widened by the parallel arrangement.

In a preferred embodiment the kinesiologic tape comprises at least one molded body having a height of 0.1 to 5 mm, preferably 0.2 to 3.5 mm, particularly preferred 0.5 to 2.5 mm. In another embodiment, the kinesiologic tape comprises at least one molded body having a width of 0.05 to 10 cm, preferably 0.1 to 9 cm, particularly preferred 0.2 to 8 cm. In a further embodiment, the kinesiologic tape comprises at least one molded body having a length of 0.05 to 10 cm, preferably 0.1 to 9 cm, particularly preferred 0.2 to 8 cm. In circular molded bodies, the width of the molded body corresponds to the diameter. In a further embodiment, the at least one molded body has a curvature of up to 0.5 cm, preferably 0.25 cm, most preferred 0.1 cm height.

In one embodiment the at least one molded body of the kinesiologic tape according to the invention is an integral part of the tape. In another embodiment, the at least one molded body is applied and/or glued onto the actual kinesiologic tape.

The actual kinesiologic tape preferably comprises a material selected from the group consisting of cotton fibers, polyamide, nylon, spandex, polyester, elastane, polyethersulfone, angora, wool and/or a combination thereof. Preferably, the tape consists of cotton fibers. Further preferred, the tape comprises 85% nylon and 15% spandex; 93% nylon, 6% polyethersulfone and 1% elastane; 44% angora and 26% wool; or 26% polyamide and 4% elastane.

Further, the actual kinesiologic tape preferably exhibits on one side a layer which is adhesive to the body, wherein this layer is preferably selected from the group consisting of an acrylic and/or zinc oxide basis.

The shape of the actual kinesiologic tape may preferably be selected regardless of the shape of the at least one molded body and the shape formed by at least two molded bodies. The shape of the actual tape is preferably selected from the group consisting of a triangle, circle, rectangle, square, ellipse, rhombus, oval, Y-shape, optionally supplemented by further lines, V-shape, S-shape, X-shape, C-shape, L-shape, T-shape, cross-shape and/or a rectangle with a plurality of incisions. Particularly preferred, several kinesiologic bands can be used so that one of the above mentioned shapes can be obtained by assembling the individual tapes. A supporting effect can also be achieved in that the tape is present in varying pre-stretched degrees.

The length of the actual kinesiologic tape can preferably be adapted to the respective body part to be treated. Furthermore, in an embodiment an adaptation to the treated body part can be obtained by using multiple kinesiologic bands of equal length In another embodiment, multiple kinesiologic bands of different length are used. In another embodiment, at least three kinesiologic bands are used, wherein at least two of these bands have the same length and at least one band has a length which is different from the length of the other two bands.

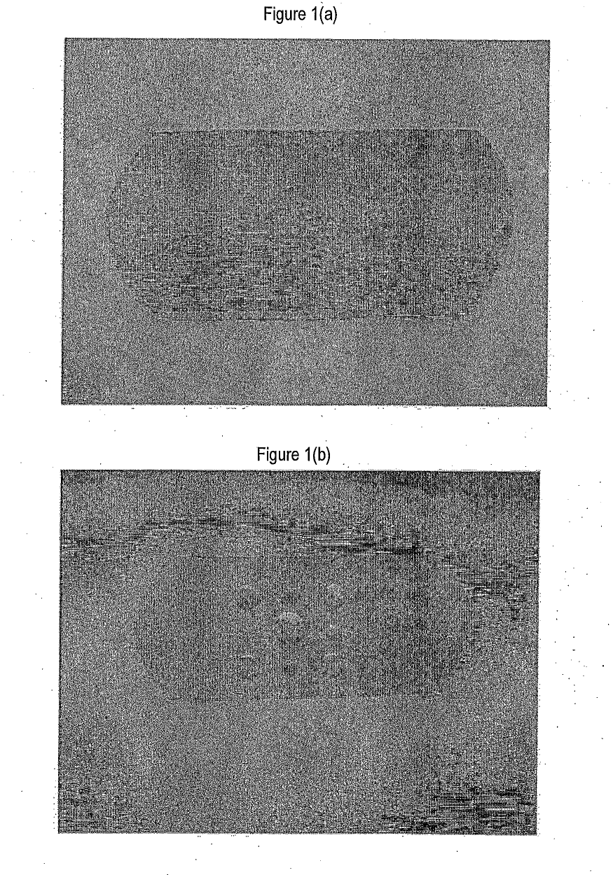

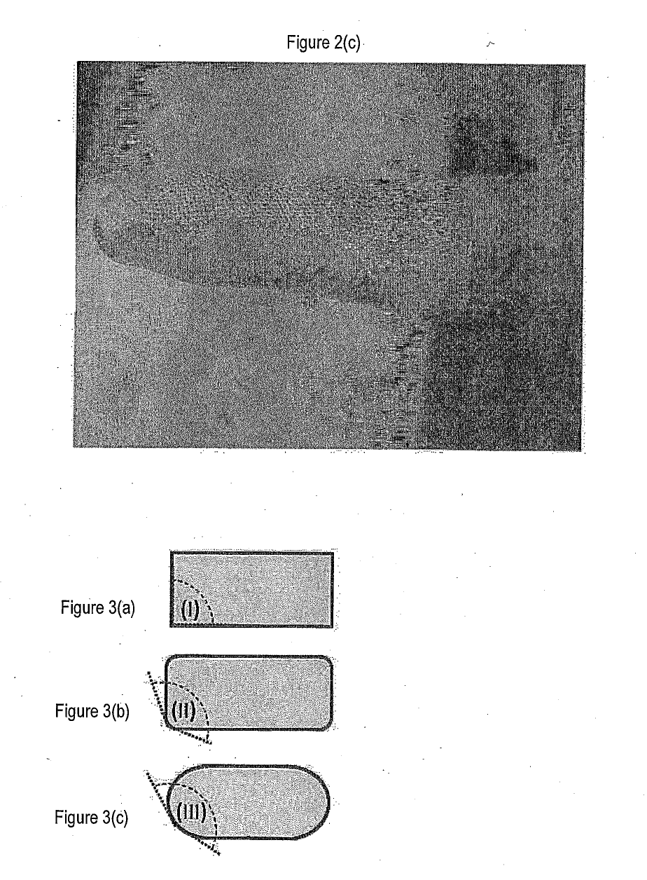

In a preferred embodiment, the kinesiologic tape of the present invention exhibit no corners with an angle of less than 90° (for determining the angle see FIG. 3), preferably less than 100°, more preferably less than 110°, and most preferred less than 120° (see FIG. 3 (b) and (c)). Particularly preferably, the tape exhibits rounded corners (see FIG. 1 and FIG. 3 (b) and (c)). It has been found that tapes that do not have corners with an angle of less than 90° have fewer contact points for peeling. This makes it possible that the kinesiologic tape of the present invention adheres to the body of the patient for a long term. The kinesiologic tape of this preferred embodiment, adheres preferably at least 14 days, more preferably at least 10 days, more preferably at least 7 days to the body of the patient.

In a particularly preferred embodiment, heat is punctually transferred to the corresponding at least one reflex point by the at least one molded body of the kinesiologic tape of the present invention. It has been shown that in addition to the synergistic effect achieved by the kinesiologic tape with at least one molded body according to the invention, a heat treatment can be implemented effectively. By the at least one molded body, the heat can directly be transferred to the reflex point, which in one embodiment may be pressure-loaded, by the at least one molded body. Especially when copper is used as material of the at least one molded body, an additional curative effect of the kinesiologic tape of the invention can be observed in a heat treatment. The heat is transferred to at least one reflex point from the far side of the body by a device selected from the group consisting of infrared heater, heating pad, hot water bottle and/or combinations thereof. It has been found advantageous that the kinesiologic tape of the invention is shaped such that the treated patient himself can generate heat by friction of the glued part of the body, which can be transmitted selectively to the at least one reflex point by the at least one molded body.

In another embodiment, cold is punctually transferred to the corresponding at least one reflex point by the at least one molded body of the kinesiologic tape of the present invention. It has been shown that in addition to the synergistic effect achieved by the kinesiologic tape with at least one molded body according to the invention, a cryotherapy can be implemented effectively. By the at least one molded body, the cold can directly be transferred to the reflex point, which in one embodiment may be pressure-loaded, by the at least one molded body. Especially when copper is used as material of the at least one molded body, an additional curative effect of the kinesiologic tape of the invention can be observed in a cryotherapy. The cold is transferred to at least one reflex point from the far side of the body by a device selected from the group consisting of cold pad, ice pack, cryosurgical device and/or combinations thereof.

In a further embodiment vibrations are punctually transferred to the at least one corresponding reflex point by the at least molded body of the kinesiologic tape according to the invention. It has been shown that in addition to the synergistic effect achieved by the kinesiologic tape with at least one molded body according to the invention the removal of blow substances in the muscles can be implemented effectively. By the at least one molded body, the vibration can directly be transferred to the reflex point, which in one embodiment may be pressure-loaded, by the at least one molded body. The vibration is transferred to at least one reflex point from the far side of the body by a device such as a Vibrax.

To further enhance the effect in a further embodiment the at least one molded body may comprise an active ingredient. Such active ingredient is preferably selected from the group consisting of anti-inflammatory, circulation-stimulating, tonus-regulating, analgesic, iontophoretic, trophic-improving, edema drainage-stimulating, heat-absorbing agents and/or a combination thereof. Particularly preferably, the active ingredient is selected from the group consisting of diclofenac, ibuprofen, essential oils, and homeopathic agents such as arnica and/or combinations thereof.

The kinesiologic tape according to the present invention is used for the treatment of diseases selected from the group consisting of cervical spondylosis (cervical spine syndrome), thoracic spine syndrome, lumbar spine syndrome (low back syndrome), shoulder-arm syndrome, biceps tendonitis, supraspinatus syndrome, epicondylitis, carpal tunnel syndrome, craniomandibular dysfunction, carpometacarpal joint, ganglion/bible bump, ISG syndrome, sciatic pain, lumboischialgia, coxarthrose, piriformis syndrome, gonarthrose, distorsion, spastic interruption, myogelosis/physical myalgia, paresis, edema, Bechterews disease, calcaneal spur, sports injuries such as muscle strains, muscle soreness, achillodynia and/or torn muscle fibers, pain such as joint pain, joint effusion, headaches, elbow pain, shoulder pain, wrist pain, upper back pain (cervical), middle (thoracic) and/or lower back pain (lumbar spine), coccyx pain, hip pain, knee pain, pain in the Achilles tendon, ankle pain, pain in the heel, pain in consequence of total joint arthroplasties (TEP). particularly in the hip or knee, internal disorders such as indigestion, abdominal pain, chronic obstipation and/or combinations thereof.

It is particularly preferred to threat the above mentioned diseases with the kinesiologic tape of the present invention in concerned humans.

In another embodiment, the present invention the kinesiologic tape of the present invention is used for cosmetic treatment. Then, preferably dark eye contours, dark eye circles, lymphedema and/or age spots can be treated.

In another embodiment, the kinesiologic tape of the present invention is used in the treatment of animals. Preferably, the animals are selected from the group consisting of horse, dog, cat, donkey, alpaca, llama, pig, cow, cattle, sheep, goat, rabbit, ferret, reindeer, Bactrian camel and dromedary. These animals may preferably have one of the diseases described above. Further preferably, the animals can also have medical conditions and/or symptoms that are selected from the group consisting of anorexia, paralysis, lameness, swaying, limping, hip dysplasia and elbow dysplasia.

In a further embodiment, the kinesiologic tape of the present invention is preferably applied to the body to be treated by an additional fixation. Such a fixation preferably supports and improves the adhesive properties of the kinesiologic tape of the present invention on the body. This makes it possible that the kinesiologic tape of the present invention adheres preferably at least 14 days, more preferably at least 10 days, most preferably at least 7 days on the patient's body.

Such additional fixation is preferably a bandage or dressing. Such bandage may preferably include elastic materials. Particularly preferably the bandage comprises materials selected from the group consisting of thermosetting plastics, thermoplastics, elastomers, epoxy resins, natural rubber, polyester resins, silicones, synthetic fibers, aramid fibers, glass fibers, nylon, Perlon, cotton, and elastane.

The additional fixation can preferably be applied to the kinesiologic tape of the present invention.

In an embodiment, the additional fixation exhibits an adhesive layer on one side. Such adhesive layer may preferably be used to adhere to the kinesiologic tape and/or the patient's skin, thereby ensuring an additional fixation. The adhesive layer preferably comprises the above-mentioned materials for adhesive layers.

In another embodiment, it is also possible that the adhesive layer is applied only to a portion of the additional fixation. Such additional fixation is preferably applied to the kinesiologic tape of the present invention in that it is enclosed by the additional fixing. The fixation itself is preferably connected with another portion of the additional fixation by the part having the adhesive layer. This embodiment is particularly suitable for use on body parts that can be enclosed by the fixation. Such body parts can preferably be selected from the group consisting of the wrist, elbow, upper arm, lower arm, trunk, head, thigh, knee, lower leg, ankle and foot. For this purpose, the additional fixation preferably has an accordingly adapted shape. It is for example possible that the fixation in total has the same dimensions as the kinesiologic tape of the present invention, but however, is a little longer. On this longer portion preferably the adhesive layer is located. Preferably, the portion on which the adhesive layer is located is narrower than the rest of the fixation. Preferably, the region of the additional fixation, which is provided with an adhesive layer, is 0.2 to 20 cm, particularly preferably 0.4 to 17 cm, most preferably 0.5 to 14 cm. The advantage of this embodiment is that the adhesive layer is applied essentially on the other side of the additional fixation (for example, when enclosing a wrist). This allows the use of adhesive layer materials with strong adhesion but probably allergic potential. Since there is no contact with the skin of the patient no allergic reactions against this adhesive material and at the same time a strong adhesion can be realized.

In another embodiment, the additional fixation is provided on one part with at least one clip or button instead of or in addition to an adhesive layer. Particularly preferably no adhesive layer is then used, whereby an allergic reaction to the components of the adhesive layer can be avoided, but nevertheless a firm fixation is guaranteed by the at least one clip or button.

A particular advantage of the kinesiologic tape of the present invention is the possibility to provide very flexible embodiments as described above. With the kinesiologic tape of the present invention it is possible to individually adjust the shape or different shapes of the at least one molded body as well as of the actual tape itself to the disease of an individual patient, as well as to the individual body shape of the patient.

In a further aspect the invention provides a kit of parts comprising a kinesiologic tape and at least a molded body, which is characterized in that the at least one molded body comprises a material which is selected from the group consisting of metals, healing stones, wood, ceramics and/or combinations thereof. In a preferred embodiment, this kit of parts can be used in the treatment of the above mentioned diseases. This kit of parts comprises at least one molded body, as described above, and a kinesiologic tape as described above.

The individual adjustment can be illustrated in the following examples:

For the treatment of spastic interruptions the kinesiologic tape of the present invention is preferably used with pointed molded bodies.

For the treatment of edema, the kinesiologic tape of the present invention is preferably used with at least one molded body having a diameter of preferably 0.1 to 0.5 cm, more preferably 0.15 to 0.2 cm.

For the treatment of calcaneal spur the kinesiologic tape of the present invention is preferably used with at least one concave molded body, wherein the calcaneal spur is encompassed by this shape of the molded body. In a further embodiment for the treatment of calcaneal spur, a plurality of molded bodies is applied to the kinesiologic tape of the present invention in a circle (O-form), wherein the tape is applied so that the calcaneal spur is located in the area enclosed by the molded bodies.

To relieve the masseter or the temporomandibular joint in craniomandibular dysfunction by application to the temporomandibular joint the kinesiologic tape of the present invention is preferably used with at least one round, flat or slightly curved molded body. The molded body preferably has a diameter of 0.3 to 1.0 cm, preferably from 0.4 to 0.9 cm, particularly preferably 0.6 to 0.8 cm. The tape length is preferably 0.5 to 20 cm. It is also possible to apply the tape on the temporomandibular joint so that multiple tape strips are glued one after the other. The tape is preferably rectangular and/or round.

For the treatment of head discomfort by application on the temples, between the eyebrows, on the eyelid and/or on the forehead, at least one round, flat or slightly curved molded body is used. The molded body preferably has a diameter of 0.3 to 1.0 cm, preferably from 0.4 to 0.9 cm, particularly preferably 0.6 to 0.8 cm. The tape length is preferably 0.5 to 20 cm. The shape of the tape for application to the temples, forehead or between the eyelids is preferably rectangular. For application on the eyelid preferably a C-shaped tape is chosen.

For the treatment of cervical syndrome with headache, dizziness, tinnitus, eye pressure and/or drawing pains in the arms by application on the torticollis the kinesiologic tape of the present invention is preferably applied on the back of the head in the direction of shoulders and/or next to or on the cervical spine. Preferably the at least one molded body has a thickness of 0.5 to 2.5 mm. The tape length is preferably 15 to 30 cm. The shape of the tape is preferably chosen from an Y-shape or a straight line. More preferably a straight tape with two parallel lines of a plurality of molded bodies is selected, wherein the lines of the molded bodies extend left and right of the cervical spine.

For the treatment of thoracic syndrome with respiratory distress and/or radiating pain the kinesiologic tape is preferably applied next to or on the thoracic spine. The tape length is preferably 10 to 40 cm. Here, preferably two essentially straight tapes according to the invention with at least three essentially linear applied molded bodies can be applied parallel to the thoracic spine. In another embodiment, only one tape is used which has two lines of the molded bodies extending parallel to the thoracic spine. Preferably, additional linear kinesiologic tapes according to the present invention can be applied straight from the shoulders to the thoracic spine. These may optionally have at least three molded bodies in a linear arrangement. In a further embodiment, both the shape of the tape as well as the shape that is formed by the molded bodies is an S-shape and/or mirror-inverted and is applied adjacent to the thoracic spine.

For the treatment of lumbar syndrome with pain, radiating pain, intervertebral disc problems and/or degenerative wear of the vertebral joints the kinesiologic tape of the present invention is preferably applied T- and/or C-shaped in the region of the lumbar spine. In one embodiment, the corresponding shapes are applied next to the lumbar spine. In a further embodiment essentially linear kinesiologic tapes according to the present invention can be applied horizontally across the region of the lumbar spine, or extend right and left of the lumbar spine.

For the treatment of knee arthritis, sports injuries of the knee, traumatic injuries to the knee and/or postoperative treatments at the knee the kinesiologic tape of the present invention is preferably applied with a molded body with a diameter of 10 cm. The application is preferably carried out with two kinesiologic tapes of the present invention each lateral and medial to the knee.

For the treatment of paralysis the kinesiologic tape of the present invention is preferably applied with at least one molded body having a diameter of preferably 0.05 to 0.5 cm, more preferably 0.15 to 0.2 cm and/or at least one pointed molded body. The tape length is preferably 30 to 50 cm.

For the treatment of carpal tunnel syndrome the kinesiologic tape of the present invention is applied in a series of two or more round-shaped molded bodies of the same or of different size. The tape length is preferably 1 to 10 cm, particularly preferably 3 to 7 cm.

For the treatment of ganglion the kinesiologic tape of the present invention is preferably applied with at least one molded body having a diameter of 1.5 cm. The molded body preferably has a thickness of 0.5 cm. The tape length is preferably 5 cm.

For the treatment of pain in the region of the arm the kinesiologic tape of the present invention is preferably applied in the region of the upper arm. The tape may preferably applied in an L-shape, an S-shape or in the shape of a plurality of parallel arranged essentially straight lines of the same or different size.

In order to support the healing of scar tissue the kinesiologic tape of the present invention is preferably applied around the scar tissue. The scar itself is preferably not glued, which means that the kinesiologic tape of the present invention is applied around the affected scar tissue.

Preferred embodiments are summarized in Table 1.

TABLE 1
possible embodiments of the invention:

| | | Shape of the molded body/ | | |
|---|---|---|---|---|
| Shape of | Shape of the | the molded bodies on the | | |
| the tape | molded body | tape | Disease | Position on the body |
| rectangular | round, slightly curved | 1 molded body applied in | dizziness, headache, | forehead |
| | | the middle | migraine, tinnitus | |
| rectangular | round, slightly curved | 1 molded body applied in | headache | between the eyebrows |
| | | the middle | | |
| rectangular | hemispherical | 1 molded body applied in | headache | between the eyebrows |
| | | the middle | | |
| round | hemispherical | 1 molded body applied in | headache | between the eyebrows |
| | | the middle | | |
| round | round, slightly curved | 1 molded body applied in | headache | between the eyebrows |
| | | the middle | | |
| rectangular | round, slightly curved | 1 molded body applied in | Craniomandibular | temporomandibular |
| | | the middle | dysfunction | joint |
| rectangular | round, slightly curved | plurality of essentially | Craniomandibular | temporomandibular |
| | | linear applied molded | dysfunction | joint |
| | | bodies | | |
| rectangular | round, slightly curved | 1 molded body applied in | headache, migraine, | temple |
| | | the middle | | |
| rectangular | hemispherical | 1 molded body applied in | headache, migraine, | temple |
| | | the middle | | |
| round | round, slightly curved | 1 molded body applied in | headache, migraine, | temple |
| | | the middle | | |
| round | round, slightly curved | 1 molded body applied in | Craniomandibular | temporomandibular |
| | | the middle | dysfunction | joint |
| C-shaped | round, slightly curved | C-shaped application of a | dark eye rings, | eye lid |
| | | plurality of molded bodies | lymphoedema | |
| essentially | round, slightly curved | plurality of essentially | dizziness, headache | extension: behind |
| linear | | linear applied molded | | one ear via neck |
| | | bodies | | to the sternum |
| Y-shaped | round, slightly curved | plurality of molded bodies | dizziness, headache | extension: behind |
| | | applied in an Y-shape | | one ear via neck |
| essentially | round, slightly curved | plurality of molded bodies | cervical spine | on cervical spine |
| linear | | in several parallel lines | syndrome and | (lines of molded |
| | | | corresponding | bodies extend next to |
| | | | symptoms | vertebral bodies) |
| essentially linear | round, slightly curved | several molded bodies | cervical spine syndrome | extension: from neck to |
| | | in line | and corresponding | shoulder |
| | | | symptoms | |
| essentially linear | round, slightly curved | several molded bodies | cervical spine syndrome | encompass the shoulder |
| | | in line | and corresponding | |
| | | | symptoms | |
| three essentially | round, slightly curved | plurality of molded bodies | thoracic spine syndrome | 1 tape vertical in the |
| linear extending | | in several parallel lines | and corresponding | thoracic spine region |
| tapes, together | | | symptoms | (lines of molded bodies |
| forming an Y-shape | | | | extend next to vertebral |
| | | | | bodies); |
| | | | | 2 tapes each from |
| | | | | shoulder to the tape |
| | | | | tape applied in the |
| | | | | middle |
| two S-shaped or | round, slightly curved | S-shaped or mirror-inverted | thoracic spine syndrome | laterally offset right |
| mirror-inverted | | S-shaped arrangement | and corresponding | and left of the |
| S-shaped | | | symptoms | vertebral spine |
| tapes | | | | |
| C-shaped | round, slightly curved | C- shaped | lumbar spine syndrome | laterally offset to the |
| | | | and corresponding | lumbar spine |
| | | | symptoms | |
| T-shaped | round, slightly curved | T- shaped | lumbar spine syndrome | laterally offset to the |
| | | | and corresponding | lumbar spine |
| | | | symptoms | |
| essentially linear | round, slightly curved | several molded bodies | lumbar spine syndrome | horizontally across the |
| | | in line | and corresponding | lumbar spine |
| | | | symptoms | |
| 2 essentially linear | round, slightly curved | several molded bodies | lumbar spine syndrome | left and right next to |
| tapes | | in line | and corresponding | the lumbar spine |
| | | | symptoms | |
| 2 essentially | round, slightly curved | V- shaped | Achillodynia | back, upper region of |
| lineartTapes, | | | | the calf (opening of |
| together | | | | the V) to |
| forming a V-shape | | | | the heel |
| round | round, slightly curved | 1 molded body applied in | tennis elbow (outward), | lower arm |
| | | the middle | golfer's elbow (inward) | |
| rectangular | oval | 1 molded body applied in | arthrosis, after | lateral knee joint |
| | | the middle | operations or trauma | |
| plurality of | round, slightly curved | Y-shaped with additional | Lumboischialgia, sports | back, upper thigh |
| essentially linear | | axial extending leg | injuries and symptoms | (opening of the V) to |
| extending tapes, | | | | the knee joint |
| together forming an | | | | |
| Y-shape with | | | | |
| additional axial | | | | |
| extending leg | | | | |
| round | round | circular | calcaneal spur | heel |
| T-shaped | round | 2 molded bodies, each at | Arthritis | finger |
| | | the wings of the T | | |
| T-shaped | round | 2 molded bodies, each at | carpal tunnel syndrome | wrist |
| | | the wings of the T | | |
| T-shaped | round | 2 molded bodies, each at | sports injuries | ankle joint |
| | | the wings of the T | | |

### SHORT DESCRIPTION OF THE FIGURES

FIG. 1: Illustration of a preferred embodiment of the kinesiologic tape of the present invention with corners, which are all greater than 90° (length of the tape in the middle: 15 cm)
(a) picture of the kinesiologic tape from the side far to the body site to be glued;
(b) picture of the kinesiologic tape from the side facing the body site to be glued (arrangement of the molded bodies made of copper in a rectangle, wherein the molded bodies have a diameter of (upper and lower row from left to right): 0.8 cm, 0.5 cm, 0.8 cm, (middle row from left to right): 0.5 cm, 1.1 cm, 0.5 cm)

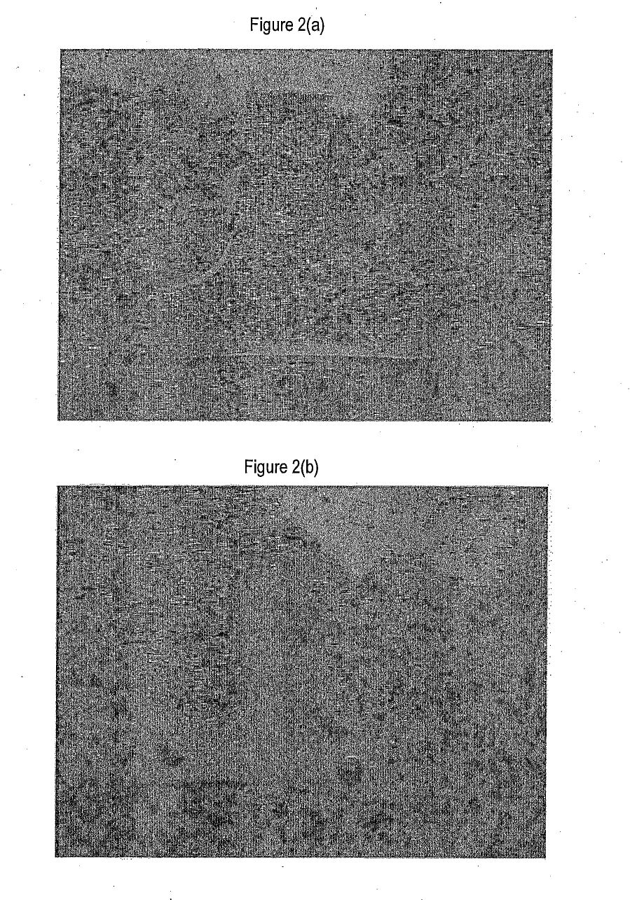

FIG. 2: Illustration of a preferred embodiment of the kinesiologic tape of the present invention for application on fingers, hands, or ankles, wherein the shape of the tape corresponds to a T (depending on the application, the tape length is 3 to 10 cm, and the width (wings) is from 3 to 10 cm)
(a) picture of the kinesiologic tape from the side far to the body site to be glued;
(b) picture of the kinesiologic tape from the side facing the body site to be glued (application of one molded body made of copper each on the wings, the diameter of the molded body: 0.5 cm)
(c) application of the kinesiologic tape on a finger

FIG. 3: Schematic illustration of the angle of the corners of the kinesiologic tape of the present invention by applying tangents
(a) kinesiologic tape, wherein the corners have an angle of 90° (I)
(b) kinesiologic tape, wherein the corners have an angle greater than 90° (II) (rounded corners)
(c) kinesiologic tape, wherein the corners have an angle greater than 120° (III) (rounded corners)

### EXAMPLES

The following Table 2 shows Examples of the results of the application of the kinesiologic tape of the present invention in the treatment of pain of different diseases. Therein the patients initially assessed their pain in a scale from 0 to 10 before the beginning of the treatment. Here, a value of 0 has the meaning of no pain, and a value of 10 indicates severe pain. Then the patients were treated with the kinesiologic tape of the present invention. 7 days after starting the treatment, the patients each assessed their pain again with the above scale from 0 to 10. During this period of 7 days, the kinesiologic tape of the present invention was not renewed.

TABLE 2
Results of the pain therapy in different diseases with the kinesiologic tape of the present invention

| | Date of the | sex of the | Date of birth of | | Assessment of pain | Assessment of pain |
|---|---|---|---|---|---|---|
| Patient | treatment | patient | the patient | Disease | before the treatment | after 7 days |
| 1 (human) | 30 Aug. 2011 | male | 09 Oct. 1942 | cervical spine syndrome after | 7 | 3 |
| | | | | accident | | |
| 2 (human) | 30 Aug. 2011 | female | 27 Jul. 1944 | lumbal spine syndrome | 9 | 1 |
| 3 (human) | 06 Sep. 2011 | female | 02 Sep. 1939 | hip TEP right | 10 | 0 |
| 4 (human) | 22 Aug. 2011 | female | 26 Sep. 1948 | protruding invertebral disc | 5 | 0 |
| 5 (human) | 22 Aug. 2011 | male | 05 Oct. 1958 | knee operation | 7 | 5 |
| 6 (human) | 02 Aug. 2011 | female | 06 Sep. 1961 | lumbar disc herniation | 8 | 3 |
| 7 (human) | 18 Aug. 2011 | female | 15 Nov. 2000 | knee pain | 8 | 1 |
| 8 (human) | 18 Aug. 2011 | male | 03 Jan. 1944 | tennis elbow | 7 | 0 |
| 9 (human) | 22 Aug. 2011 | male | 10 Nov. 1941 | apoplexy right arm spasticity | 8 | 3 |
| 10 (human) | 27 Aug. 2011 | female | 19 May 1954 | disc herniation | 6 | 2 |
| 11 (human) | 23 Aug. 2011 | female | 25 Apr. 1975 | lumbal spine syndrome | 7 | 0 |
| 12 (human) | 25 Aug. 2011 | male | 19 Aug. 1939 | shoulder-arm syndrome | 6 | 4 |
| 13 (human) | 25 Aug. 2011 | female | 01 Jul. 1987 | lumbal spine syndrome | 10 | 0 |
| 14 (human) | 25 Aug. 2011 | male | 10 Apr. 1963 | cervical spine syndrome | 2 | 0 |
| 15 (human) | 01 Sep. 2011 | male | 15 Jun. 1934 | lumbal spine syndrome after | 8 | 1 |
| | | | | appr. 5 years | | |
| 16 (human) | 29 Aug. 2011 | female | 23 Dec. 1952 | protruding invertebral disc | 8 | 0 |
| 17 (human) | 22 Aug. 2011 | male | 03 Feb. 1974 | lumbal spine syndrome | 8 | 1 |
| 18 (horse) | 02 Sep. 2011 | female | | difficulties in walking | — | was able to walk |
| | | | | | | again |
| 19 (human) | 02 Sep. 2011 | female | 05 Nov. 1976 | tingling in fingers right, arm | 8 | 0 |
| 20 (human) | 02 Sep. 2011 | female | 08 Feb. 1939 | knee TEP left | 5 | 0 |

1-10. (canceled)

11. A kinesiologic tape with at least one molded body, characterized in that the at least one molded body comprises a material which is selected from the group consisting of metals, healing stones, wood, ceramics and combinations thereof.

12. The kinesiologic tape according to claim 11, characterized in that the at least one molded body is formed such that it can apply at least one of a planar pressure and a punctual pressure to at least one reflex point.

13. The kinesiologic tape according to claim 11, characterized in that the material of the at least one molded body has a thermal conductivity of 200 to 400 W/(m·K).

14. The kinesiologic tape according to claim 12, characterized in that the material of the at least one molded body has a thermal conductivity of 200 to 400 W/(m·K).

15. The kinesiologic tape according to claim 11, characterized in that the at least one molded body has a shape selected from the group consisting of sphere, sphere chain, ring, hemisphere, ellipsoid, rotational ellipsoid, paraboloid, rotational paraboloid, hyperboloid, oloid, polyhedron, tetrahedron, cube, cuboid, octahedron, icosahedron, dodecahedron, cuboctahedron, rhombic dodecahedron, icosidodecahedron, rhombic triaktontahedron, deltahedron, pyramid, prism, antiprism, truncated pyramid, torus, cylinder, cone, truncated cone, geodesic dome and combinations thereof.

16. The kinesiologic tape according to claim 12, characterized in that the at least one molded body has a shape selected from the group consisting of sphere, sphere chain, ring, hemisphere, ellipsoid, rotational ellipsoid, paraboloid, rotational paraboloid, hyperboloid, oloid, polyhedron, tetrahedron, cube, cuboid, octahedron, icosahedron, dodecahedron, cuboctahedron, rhombic dodecahedron, icosidodecahedron, rhombic triaktontahedron, deltahedron, pyramid, prism, antiprism, truncated pyramid, torus, cylinder, cone, truncated cone, geodesic dome and combinations thereof.

17. The kinesiologic tape according to claim 13, characterized in that the at least one molded body has a shape selected from the group consisting of sphere, sphere chain, ring, hemisphere, ellipsoid, rotational ellipsoid, paraboloid, rotational paraboloid, hyperboloid, oloid, polyhedron, tetrahedron, cube, cuboid, octahedron, icosahedron, dodecahedron, cuboctahedron, rhombic dodecahedron, icosidodecahedron, rhombic triaktontahedron, deltahedron, pyramid, prism, antiprism, truncated pyramid, torus, cylinder, cone, truncated cone, geodesic dome and combinations thereof.

18. The kinesiologic tape according to claim 11, characterized in that at least one of heat and cold is punctually transferred to a corresponding at least one reflex point by the at least one molded body.

19. The kinesiologic tape according to claim 12, characterized in that at least one of heat and cold is punctually transferred to a corresponding at least one reflex point by the at least one molded body.

20. The kinesiologic tape according to claim 13, characterized in that at least one of heat and cold is punctually transferred to a corresponding at least one reflex point by the at least one molded body.

21. The kinesiologic tape according to claim 11, characterized in that the molded body is coated with an active ingredient selected from the group consisting of anti-inflammatory, circulation-stimulating, tonus-regulating, analgesic, iontophoretic, trophic-improving, edema drainage-stimulating, heat-absorbing agents and combinations thereof.

22. The kinesiologic tape according to claim 12, characterized in that the molded body is coated with an active ingredient selected from the group consisting of anti-inflammatory, circulation-stimulating, tonus-regulating, analgesic, iontophoretic, trophic-improving, edema drainage-stimulating, heat-absorbing agents and combinations thereof.

23. The kinesiologic tape according to claim 13, characterized in that the molded body is coated with an active ingredient selected from the group consisting of anti-inflammatory, circulation-stimulating, tonus-regulating, analgesic, iontophoretic, trophic-improving, edema drainage-stimulating, heat-absorbing agents and combinations thereof.

24. The kinesiologic tape according to claim 11 for the treatment of diseases selected from the group consisting of cervical spondylosis (cervical spine syndrome), thoracic spine syndrome, lumbar spine syndrome (low back syndrome), shoulder-arm syndrome, biceps tendonitis, supraspinatus syndrome, epicondylitis, carpal tunnel syndrome, craniomandibular dysfunction, carpometacarpal joint, ganglion/bible bump, ISG syndrome, sciatic pain, lumboischialgia, coxarthrose, piriformis syndrome, gonarthrose, distorsion, spastic interruption, myogelosis/physical myalgia, paresis, edema, Bechterews disease, calcaneal spur, sports injuries such as muscle strains, muscle soreness, achillodynia and/or torn muscle fibers, pain such as joint pain, joint effusion, headaches, elbow pain, shoulder pain, wrist pain, upper back pain (cervical), middle (thoracic) back pain, lower back pain (lumbar spine), coccyx pain, hip pain, knee pain, pain in the Achilles tendon, ankle pain, pain in the heel, internal disorders such as indigestion, abdominal pain, chronic obstipation and combinations thereof.

25. The kinesiologic tape according to claim 12 for the treatment of diseases selected from the group consisting of cervical spondylosis (cervical spine syndrome), thoracic spine syndrome, lumbar spine syndrome (low back syndrome), shoulder-arm syndrome, biceps tendonitis, supraspinatus syndrome, epicondylitis, carpal tunnel syndrome, craniomandibular dysfunction, carpometacarpal joint, ganglion/bible bump, ISG syndrome, sciatic pain, lumboischialgia, coxarthrose, piriformis syndrome, gonarthrose, distorsion, spastic interruption, myogelosis/physical myalgia, paresis, edema, Bechterews disease, calcaneal spur, sports injuries such as muscle strains, muscle soreness, achillodynia and/or torn muscle fibers, pain such as joint pain, joint effusion, headaches, elbow pain, shoulder pain, wrist pain, upper back pain (cervical), middle (thoracic) back pain, lower back pain (lumbar spine), coccyx pain, hip pain, knee pain, pain in the Achilles tendon, ankle pain, pain in the heel, internal disorders such as indigestion, abdominal pain, chronic obstipation and combinations thereof.

26. The kinesiologic tape according to claim 13 for the treatment of diseases selected from the group consisting of cervical spondylosis (cervical spine syndrome), thoracic spine syndrome, lumbar spine syndrome (low back syndrome), shoulder-arm syndrome, biceps tendonitis, supraspinatus syndrome, epicondylitis, carpal tunnel syndrome, craniomandibular dysfunction, carpometacarpal joint, ganglion/bible bump, ISG syndrome, sciatic pain, lumboischialgia, coxarthrose, piriformis syndrome, gonarthrose, distorsion, spastic interruption, myogelosis/physical myalgia, paresis, edema, Bechterews disease, calcaneal spur, sports injuries such as muscle strains, muscle soreness, achillodynia and/or torn muscle fibers, pain such as joint pain, joint effusion, headaches, elbow pain, shoulder pain, wrist pain, upper back pain (cervical), middle (thoracic) back pain, lower back pain (lumbar spine), coccyx pain, hip pain, knee pain, pain in the Achilles tendon, ankle pain, pain in the heel, internal disorders such as indigestion, abdominal pain, chronic obstipation and combinations thereof.

27. A kit of parts comprising a kinesiologic tape and at least one molded body, characterized in that the at least one molded body comprises a material which is selected from the group consisting of metals, healing stones, wood, ceramic and combinations thereof.

28. The kit of parts according to claim 27 for the treatment of diseases selected from the group consisting of cervical spondylosis (cervical spine syndrome), thoracic spine syndrome, lumbar spine syndrome (low back syndrome), shoulder-arm syndrome, biceps tendonitis, supraspinatus syndrome, epicondylitis, carpal tunnel syndrome, craniomandibular dysfunction, carpometacarpal joint, ganglion/bible bump, ISG syndrome, sciatic pain, lumboischialgia, coxarthrose, piriformis syndrome, gonarthrose, distorsion, spastic interruption, myogelosis/physical myalgia, paresis, edema, Bechterews disease, calcaneal spur, sports injuries such as muscle strains, muscle soreness, achillodynia and/or torn muscle fibers, pain such as joint pain, joint effusion, headaches, elbow pain, shoulder pain, wrist pain, upper back pain (cervical), middle (thoracic) back pain, lower back pain (lumbar spine), coccyx pain, hip pain, knee pain, pain in the Achilles tendon, ankle pain, pain in the heel, internal disorders such as indigestion, abdominal pain, chronic obstipation and combinations thereof.

29. The kit of parts according to claim 27, wherein the at least one molded body is formed such that it can apply at least one of a planar pressure and a punctual pressure to at least one reflex point.

30. The kit of parts according to claim 28, wherein the at least one molded body is formed such that it can apply at least one of a planar pressure and a punctual pressure to at least one reflex point, wherein the material of the at least one molded body has a thermal conductivity of 200 to 400 W/(m·K).
